(19) United States
(12) Patent Application Publication
(10) Pub. No.: US 2003/0017136 A1
(43) Pub. Date: Jan. 23, 2003

(54) Pharmaceutical compositions comprising vitamin B12 and interferon-beta for treating multiple sclerosis

(76) Inventors: Tony F. Cruz, Toronto (CA); Alexandra Pastrak, Toronto (CA)

Correspondence Address: Mary Ann Dillahunty, Esq., BURNS, DOANE, SWECKER & MATHIS, L.L.P., P.O. Box 1404, Alexandria, VA, 22313-1404, US

(21) Appl. No.: 09/971,068

(22) Filed: Oct. 3, 2001

Related U.S. Application Data
(63) Continuation-in-part of application No. 09/908,298, filed on Jul. 17, 2001.

(51) Int. Cl. A61K 38/21; A61K 31/714

(52) U.S. Cl. 424/85.6; 514/52

(57) ABSTRACT

Pharmaceutical compositions for treating multiple sclerosis are disclosed comprising an amount of vitamin B12, vitamin B12 analogues, vitamin B12 derivatives, vitamin B12 conjugates or mixtures thereof in combination with interferon-beta. Vitamin B12 or its analogues, derivatives, conjugates or mixtures thereof are administered separately or in combination with interferon-beta to have a synergistic effect resulting in an enhanced therapeutic efficacy for treating multiple sclerosis.

### FIELD OF THE INVENTION

This application is a continuation-in-part of U.S. patent application Ser. No. 09/908,298 filed on Jul. 17, 2001. The present invention relates to the treatment of multiple sclerosis with vitamin B12, vitamin B12 analogues, derivatives or conjugates thereof in combination with interferon-beta.

### BACKGROUND OF THE INVENTION

Multiple sclerosis is a multifactorial inflammatory disease of the human central nervous system resulting in the slowing of electrical conduction along the nerve. It is a progressively, debilitating disease that manifests itself in the prime of peoples' lives, usually between the ages of 20 and 40. Although not fatal, the disease is unpredictable and can lead to blurred vision, loss of balance, poor coordination, slurred speech, tremors, numbness, extreme fatigue, even paralysis and blindness. It is estimated that close to a third of a million people in the United States have multiple sclerosis. It is more prevalent in northern latitudes where it affects roughly 140 per 100,000 people (0.14%). Twice as many women as men have multiple sclerosis. The cause of multiple sclerosis is unknown, although genetic and environmental factors are associated with higher risk.

Multiple sclerosis results from inflammation and breakdown in the myelin surrounding the nerve fibres of the central nervous system. Once the myelin is destroyed, it is replaced by sclerotic patches or plaques which can appear in multiple locations within the central nervous system. Hence the name “multiple sclerosis”. The disease is further characterized by an increase in the infiltration of inflammatory cells, loss of oligodendrocytes, and increased gliosis (For review see Amit et al., 1999; Pouly et al., 1999; Steimnan et al., 1993; Miller et al., 1994). Although the pathology is well established, the cause of the disease is not well elucidated. It is believed to be an autoimmune disorder where the immune system launches an attack against its own tissues, the main target being the myelin sheath.

Over the last decade there has been a broad approach to multiple sclerosis treatments. These include antigen-specific immunosuppressive compounds, general immunosuppressive compounds, antiproliferative compounds, cytokine compounds, remyelination compounds and compounds to improve nerve conduction (For review see Polman et al, 2000; Tselis et al., 1999; Rudick et al., 1999; Rudick et al, 1997).

The principal medications in the past include steroids possessing anti-inflammatory activity, including adrenocorticotropic hormone, prednisone, prednisolone, methylprednisolone, betamethasone and dexamethasone. Although these steroids reduce the severity and duration of the attacks in some patients, the drawback is that they do not affect the course of the disease over time.

More recently, the major drugs used for the treatment of multiple sclerosis include the beta-interferon compounds: Avonex™, Betaseron™ and Rebif™. These treatments have been shown to reduce the number of exacerbations, formation of plaques and slow the progression of the physical disability (Yong et al.,1998; Chofflon et al., 2000; Weinstock-Guttman et al., 2000). Although the mechanism of action in uncertain, it is thought that the interferon beta suppresses the immune system. However, at the dosages used and the length of time required for treatment, the interferon beta compounds produce adverse effects. These include local injection reactions, flu-like syndrome and depression. In some patients reduction in dosage or discontinuation of the drug may be required. Moreover, up to 40% of the patients develop neutralizing antibodies to interferon-beta.

An additional agent on the market is Copaxone™, a synthetic copolymer with some immunological similarities to myelin basic protein that has been shown to suppress the progression of the disease by inhibiting the immune system (Johnson et al., 1995; Johnson et al., 1998). Again, because of the dosages required and the length of treatment, many adverse reactions are manifested. The side effects of this compound are similar to those observed with interferon therapies.

There are a number of emerging new compounds for treating multiple sclerosis which are either in clinical trials or in early research development. These include the development of vaccines for multiple sclerosis using myelin proteins, or treating with myelin basic protein or fragments of this protein to decrease the attack of the immune system on myelin (U.S. Pat. No. 6,036,957; W.O. Pat. No. 9913904; U.S. Pat. No. 5,858,980; U.S. Pat. No. 5,948,764; W.O. Pat. No. 9845327). Other compounds include the use of antibodies to cytokines such as anti-TNF (tumor necrosis factor) antibodies which inhibit inflammatory responses as well as tissue destruction (U.S. Pat. No. 5,888,511; U.S. Pat. No. 5,958,409; U.S. Pat. No. 6,143,866). Interleukin-4 is also capable of inhibiting demyelination and improving the clinical course of disease in mouse models of multiple sclerosis (Xu et al., 1999). Although these new compounds are promising, they have multiple effects on the immune system, such as increased number of activated T cells and enhanced proliferation and cytokine secretion. Therefore, they may be problematic in treating established autoimmune diseases such as multiple sclerosis.

Anti-proliferative agents commonly used in cancer therapy have been used to treat multiple sclerosis. Currently paclitaxel in micelles, a microtubule stabilizing agent is in phase II clinical trial (Cao et al., 2000). This agent inhibits demyelination and destroy T-cells. Other agents inhibit cell migration and invasion such as LeukArrest™ and Antegren™ which are related to targets such as alpha-4 integrin and intracellular adhesion molecule respectively (U.S. Pat. No. 5,837,822). These are just a few of the therapies targeted to adhesion molecules and cell migration (U.S. Pat. No. 6,184,223; W.O. Pat. No. 9916791). Several agents have been used to improve nerve conduction resulting in increased motor activity and reducing disability. These include agents such as Provigil™ which is used to decrease multiple sclerosis fatigue (W.O. Pat. No. 0112170 A2). Again, although these new compounds are promising, they have not yet demonstrated their efficacy and safety in long-term clinical studies.

Vitamin D3 has been shown to inhibit the course of disease in the experimental autoimmune encephalomyelitis (EAE) mouse model, cytokine production and T-cell response. There are several reports and patents on this observation, but no clinical data as of yet (U.S. Pat. No. 5,716,946; Nataf et al., 1996; Mattner et al., 2000).

The choice of mouse model is crucial in the assessment of new therapeutic drugs. The experimental autoimmune encephalomyelitis (EAE) mouse model is the model most often used in multiple sclerosis drug discovery. The model is produced by immunizing susceptible rodent strains with central nervous system proteins which induce multiple sclerosis-like paralytic disease. The mouse develops inflamed multiple sclerosis and EAE lesions which contain infiltrated CD4 T-cells that respond to self-antigens. The CD4 T-cells produce pro-inflammatory cytokines (interleukin-2, interferony and interferonα) that activate macrophages to produce inflammatory cytokines (interleukin-1β, interleukin-6, interleukin-8) and interleukin-12. Interleukin-12 further induces production of interferony. Thus a cycle is formed, which results in a demyelinating attack on the central nervous system, mimicking multiple sclerosis. Recently, a new transgenic mouse model for multiple sclerosis was developed, by introducing multiple cDNA copies of DM20 an alternatively spliced variant of PLP. This transgenic mouse model, designated ND4, expresses DM20 at a high level resulting in structurally unstable myelin sheaths that spontaneously demyelinate after a period of normal growth, usually after 3 months of age. Whereas the EAE model provides an autoimmune model, the demyelinating transgenic mouse model (ND4) provides a genetic model of spontaneous demyelination, which is a critical component of multiple sclerosis. There is therefore a need to study the effect of different compounds for treating multiple sclerosis in a demyelinating transgenic mouse model.

Vitamin B12 is thought to be necessary for the function of the central nervous system including the production of myelin. (Watanabe et al., 1994; Hajime et al., 1987). The level of vitamin B12 has been shown to be decreased in patients with multiple sclerosis and its deficiency was associated with signs of demyelination However, studies in humans did not show efficacy of vitamin B12 alone in the treatment of multiple sclerosis. Although EAE model is the traditional model used to identify new therapies tested, many compounds that are effective in EAE are not effective in clinic. There is therefore a need to test the efficacy of vitamin B12 and pharmaceutical compositions including vitamin B12 in a demyelinating ND4 mouse model that closely mimics the pathologies of the human disease instead of an EAE model.

There is a need for the development of superior treatments for multiple sclerosis. Due to the nature of these diseases, patients undergo long term treatments and often experience negative effects. There is a need to develop combination therapies for the treatment of multiple sclerosis wherein the combined elements have a synergistic effect. There is a further need for combination therapies that permit the use of lower dosages of the compounds without loss of therapeutic efficacy.

### SUMMARY OF THE INVENTION

The present invention provides pharmaceutical compositions for treating multiple sclerosis, methods of treating multiple sclerosis with those pharmaceutical compositions and use of the pharmaceutical compositions to treat multiple sclerosis. The pharmaceutical composition comprises vitamin B12 and interferon-beta for the treatment of multiple sclerosis. The invention also provides a pharmaceutical composition that includes analogues, derivatives or conjugates of vitamin B12 in combination with interferon-beta, interferon-beta analogues, interferon-beta derivatives and/or interferon-beta conjugates for the treatment of multiple sclerosis.

According to one aspect of the present invention, there is provided a pharmaceutical composition for the treatment of multiple sclerosis comprising: (i) a first compound selected from the group consisting of: vitamin B12, analogues of vitamin B12, derivatives of vitamin B12, conjugates of vitamin B12 and mixtures thereof; and (ii) a second compound selected from the group consisting of interferon-beta, interferon-beta analogues, interferon-beta derivatives, interferon-beta conjugates and/or mixtures thereof.

According to another aspect of the present invention, there is provided a method of treating multiple sclerosis comprising administering to a patient a pharmaceutical composition comprising (i) a first compound selected from the group consisting of: vitamin B12, analogues of vitamin B12, derivatives of vitamin B12, conjugates of vitamin B12 and mixtures thereof; and (ii) a second compound selected from the group consisting of interferon-beta, interferon-beta analogues, interferon-beta derivatives, interferon-beta conjugates, and mixtures thereof.

According to another aspect of the present invention, there is provided a method of treating multiple sclerosis comprising (i) administering to a patient a pharmaceutical composition comprising a first compound selected from the group consisting of: vitamin B12, analogues of vitamin B12, derivatives of vitamin B12, conjugates of vitamin B12 and mixtures thereof; and (ii) administering to a patient a second pharmaceutical composition comprising a second compound selected from the group consisting of interferon-beta, interferon-beta analogues, interferon-beta derivatives, interferon-beta conjugates, and mixtures thereof.

According to another aspect of the present invention, there is provided a use of vitamin B12, vitamin B12 analogues, vitamin B12 derivatives, vitamin B12 conjugates and/or mixtures thereof the treatment of multiple sclerosis in combination with interferon-beta and/or interferon-beta analogues, interferon-beta derivatives, interferon-beta conjugates, mixtures thereof for the treatment of multiple sclerosis.

### DESCRIPTION OF THE DRAWINGS

FIG. 1 is a graph showing the effect of vitamin B12, interferon-beta and the synergistic combination of vitamin B12 and interferon-beta on mice in a ND4 mouse model;

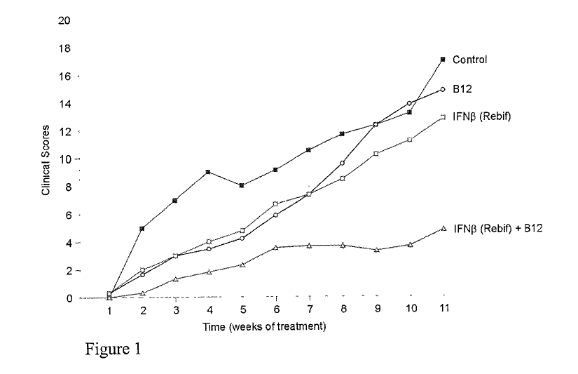

FIG. 2 is an electron micrograph showing GFAP staining in normal, ND4 untreated animals, interferon beta treated transgenic animals, and interferon-beta and vitamin B12 treated transgenic animals; and

FIG. 3 is a bar graph depicting total relative GFAP in brain homogenates;

### DEFINITIONS

CFA is complete Freunds' adjuvant.

DM20 is an isoprotein proteolipid protein. It is a major integral membrane protein of the central nervous system (CNS). DM20 is normally expressed in early (post-natal) stages of growth.

EAE (Experimental autoimmune encephalomyelitis) is a mouse model; the immunosuppressive mouse model for multiple sclerosis.

Interferon beta analogues, derivatives and conjugates: Interferon-beta genes may be altered by, for example, oligo-nucleotide directed mutagenesis to produce interferon-beta analogues thereof, such as the human recombinant cystein depleted or cystein replaced analogues. Further, identity or location of more than one amino acid may be changed by targeted mutagenesis. The primary amino acid sequence of the protein may be augmented by glycosylation or by other supplementary molecules such as lipids, phosphate, and acetyl groups. Further, individual amino acids residues in the chain may be modified by oxidation, reduction, or other derivatization. The interferon beta protein may be cleaved to obtain the fragments which retain activity. The whole protein or its fragments can be fused with other peptides and proteins such as immunoglobulins and other cytokines. Interferon beta conjugates may represent, for example, a composition comprising interferon beta coupled to a non-naturally occurring polymer comprising a polyalkylene glycol moiety.

MBP is myelin basic protein.

ND4: The transgenic mouse model for multiple sclerosis, produced by transformation with multiple copies of DM20; the genetic mouse model for multiple sclerosis.

PBS (Phosphate buffer saline) is an injectable solution that serves as a negative control because it does not have any physiological or therapeutic effects.

PLP is an isoprotein proteolipid protein. PLP becomes predominant in the adult.

PTX (pertussis toxin) is the major protein toxin produced by virulent strains of Bordetella pertussis, the organism that causes whooping cough. Pertussis toxin (PTX) is a potent ancillary adjuvant that primes macrophages used to elicit several different autoimmune diseases, including experimental allergic encephalomyelitis (EAE).

SJL/J is a mouse model. Female SJL/J mouse have increased susceptibility to development of autoimmune disease. SJL/J transgenic mouse strain is susceptible to induction of experimental allergic encephalomyelitis (EAE) (more susceptible to development of EAE than most other mouse strains). Tumor development as well as autoimmunity in this mouse may result from an effective amplification of the immune response.

### DETAILED DESCRIPTION OF THE INVENTION

The present invention, employs a demyelinating transgenic mouse model (ND4) for multiple sclerosis to investigate the effects of vitamin B12 alone and in combination with interferon-beta. It is observed that in a demyelinating mouse model, similar in pathology to the human disease, vitamin B12 alone shows efficacy during the early stages of the disease and a synergistic effect in combination with interferon-beta for treating multiple sclerosis throughout the course of disease.

The present invention includes a method of treating multiple sclerosis in patients by administering an amount of vitamin B12, or its analogues, derivatives, conjugates and mixtures thereof, in combination with interferon-beta. Vitamin B12 or its analogues, derivatives, conjugates or mixtures thereof, can be administered separately or in combination with interferon-beta to act in synergy and to enhance the efficacy of interferon-beta in the treatment of multiple sclerosis in patients compared to when it is administered alone.

The present invention further includes a pharmaceutical composition for the treatment of multiple sclerosis comprising a first compound that is at least one of vitamin B12, vitamin B12 analogues, vitamin B12 derivatives, vitamin B12 conjugates or mixtures thereof and a second compound being interferon-beta. This second compound can be also be interferon-beta conjugates, interferon-beta derivatives, interferon-beta analogues. The second compound can also include mixtures thereof.

The present invention includes methods of treatment for multiple sclerosis. The first method of treatment, is the administration of a pharmaceutical composition including both vitamin B12 and interferon-beta. An alternate method of treatment includes the step of the administration of a pharmaceutical composition including at least one of vitamin B12, vitamin B12 conjugates, vitamin B12 analogues, vitamin B12 derivatives or mixtures thereof followed by the step of the administration of a second pharmaceutical composition including at least one of interferon-beta, interferon-beta conjugates, interferon-beta derivatives, interferon-beta analogues or mixtures thereof. Optionally, the administration of the pharmaceutical compositions occurs separately and in rapid succession. The pharmaceutical compositions may comprise pharmaceutically acceptable carriers and adjuvants. Optionally, the pharmaceutical compositions are injectable formulations.

Vitamin B12 and its analogues, derivatives or conjugates thereof include the following compounds: cyanocobalamin (CN-Cb1), aquacobalamin, adenosylcobalamin, methylcobalamin, hydroxycobalamin, cyanocobalamin carbanalide, and 5-o-methylbenzylcobalmin [(5-OmeB-za)CN-Cb1] as well as the desdimethyl, monoethylamide and the methylamide analogues of all of the above. Also included are the various analogues and homologues of cobamamide such as coenzyme B12 and 5-deoxydenosylcobalamin. Other analogues include chlorocobalamin, sulfitocobalamin, nitrocobalamin, thiocyanatocobalamin, benzimidazole derivatives such as 5,6-dichlorobenzimidazole, 5-hydroxybenzimidazole, trimethylbenzimidazole, as well as adenosylcyanocobalamin [(Ade) CN-Cb1], cobalamin lactone, cobalamin lactam and the anilide, ethylamide, monocarboxylic and dicarboxylic acid derivatives of vitamin B12 or its analogues. Preferred derivatives of vitamin B12 include the mono-, di- and tricarboxylic acid derivatives or the proprionamide derivatives of vitamin B12. In addition, the compositions include polymers of these analogues or vitamin B12 conjugated to other molecules or encapsulated. Mixtures of the above compounds can also be employed.

Interferon-beta in many different forms is employed according to the present invention. These forms include Avonex™, Betaseron™ and Rebif™. Analogues or derivatives of interferon-beta include pegylated, polymerized and dimerized forms of interferon-beta, interferon-beta conjugated to carriers, or as oral, inhalant, topical and injectable compositions. Alternatively, agents that induce interferon-beta production or mimic the action of interferon-beta may be employed according to the present invention.

The dosages used for each interferon-beta compound are similar to those dosages known to those skilled in the art and used in pre-clinical and clinical studies. The concentrations may be lower than the currently used dosages as the combination of these agents with vitamin B12, vitamin B12 analogues and vitamin B12 derivatives increases efficacy of these agents. Indeed, vitamin B12, vitamin B12 analogues, vitamin B12 derivatives and vitamin B12 conjugates may be combined with interferon-beta compounds with the objective to reduce the dosages of the interferon-beta compounds, in order to achieve both effective treatment and to lessen the negative effects of the interferon-beta compounds.

The dosages for interferon-beta range from 20 μg to 250 μg. The main differences between Avonex™, Betaseron™ and Rebif™ are the amount of interferon-beta given and the route and frequency of administration. Avonex™ is preferably administered in the amount of 30 μg by intramuscular injection once weekly; Betaseron™ is preferably administered in the amount of 250 μg by subcutaneous injection every other day; and Rebif™ is preferably administered in the amount of 22 μg by subcutaneous injection three times a week.

The preferred dosage of vitamin B12 or its analogues, derivatives or conjugates thereof for the present invention is the maximum that a patient can tolerate and not develop any serious complications. To date no toxicity has been found in humans or animals, even at concentrations of 15 mg/kg in mice. Human dosages have been as high as 10 to 60 mg daily for long periods. Experimental data suggests that daily dosages between 10 mg to 1000 mg of vitamin B12 or its analogues, derivatives, conjugates, or mixtures thereof can be used in combination with other compounds listed above administered separately or in combination or both. Most preferably the dosage in humans is 2 to 3 g daily. Preferably, the dosage is within the range of 2-5 g daily.

The following are examples of acceptable regimens:

1. daily, weekly or monthly mixtures of vitamin B12 and vitamin B12 analogues, vitamin B12 derivatives, vitamin B12 conjugates or mixtures thereof in combination with interferon-beta for the effective treatment of multiple sclerosis;

2. daily, weekly or monthly treatments with interferon-beta and vitamin B12, or vitamin B12 analogues, vitamin B12 derivative, vitamin B12 conjugates or mixtures thereof administered separately either daily, weekly or monthly;

3. daily, weekly or monthly mixtures of interferon-beta and vitamin B12, vitamin B12 analogues, vitamin B12 derivatives, vitamin B12 conjugates or mixtures thereof, as well as adjunct administration of daily or weekly doses of vitamin B12 or vitamin B12 analogues, vitamin B12 derivatives, vitamin B12 conjugates or mixtures thereof.

Compositions including different proportions of analogues of vitamin B12 have an enhanced effect for treating multiple sclerosis. These compositions including different proportions of analogues of vitamin B12 act synergistically with interferon-beta and are effective for treating multiple sclerosis. In addition, the vitamin B12 composition may consist of mixtures of different B12 analogs or derivatives in combination or administered separately with interferon-beta.

Administration of a combination including vitamin B12 and interferon-beta results in enhanced efficacy for treating multiple sclerosis in comparison to interferon-beta alone.

Vitamin B12 can be also applied as adjunct therapy to interferon-beta to treat multiple sclerosis. Adjunct therapy involves the administration of vitamin B12 and interferon-beta at different times and separate administration regimens.

### EXAMPLES

The examples below are designed to demonstrate but not limit the embodiments of the present invention.

### Example 1

Effect of Vitamin B12, Interferon-beta and Combination of Vitamin B12 With Interferon Beta in ND4 Mouse Model

The ND4 transgenic mouse was produced by introducing 70 copies of the cDNA for DM20, an alternatively spliced variant of the PLP. The transgenic ND4 mouse continuously expresses DM20 at a high rate resulting in structurally unstable axons that spontaneously demyelinate after a period of normal growth (Mastronardi et al, 1995). This model is a slow progressive model where the animals demonstrate symptoms in young adults at approximately 3 months of age. The severity of the clinical signs increase until a maximum around 6 months with animals dying around 8 to 9 months of age. The clinical signs assessed include general shaking, seizures, head jerk, hind limb and tail shiver, wobbly gait and limp tail. The scale of zero (absence) to four (constant and uncontrollable movements) was used for each of the clinical signs.

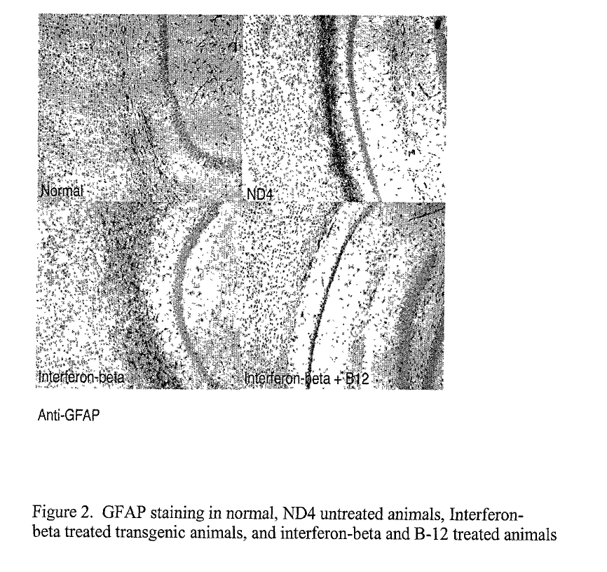

The ND4 transgenic mice receive one of the following treatments through intraperitoneal injections: 1) Interferon-β1a (Rebif™) at a dose of 5000 IU, three times per week; 2) vitamin B12 at a dose of 15 mg/kg once a week and a combination of Interferon-β1a (Rebif™) at a dose of 5000 IU, three times a week; and 3) vitamin B12 at a dose of 15 mg/kg once a week, as separate injections. All treatments start when the mice reach 2.5 months of age (2.5 months of age correspond to 0 weeks in FIG. 2) at which time signs of demyelinating disease are evident.

Treatment of the ND4 mice with the vitamin B12 alone showed attenuation of multiple sclerosis symptoms early in disease, suggesting that vitamin B12 is an effective agent in ameliorating disease symptoms at early stages (FIG. 2). As shown in FIG. 2, interferon-beta treatment alone decreases the disease activity in this model. However, the combination therapy of vitamin B12 with the Interferon-beta is approximately 60% more effective in attenuation of clinical signs compared to when interferon-beta is used alone, demonstrating a synergistic effect of interferon-beta and vitamin B12 on decreasing clinical signs of multiple sclerosis in the ND4 model.

### Example 2

Brain Immunohistochemistry—Glial Fibrillary Acidic Protein (GFAB) Staining for Astrocytes

The brain region of hippocampus, adjacent to the dentate gyrus was fixed in formalin, paraffin-embedded and sectioned at 5 μm. Immunohistochemistry with anti-GFAP anitibody was used to stain for GFAP. Glial fibrillary acidic protein is a protein expressed by astrocytes (particularly reactive astrocytes) and is used as a marker for these cells.

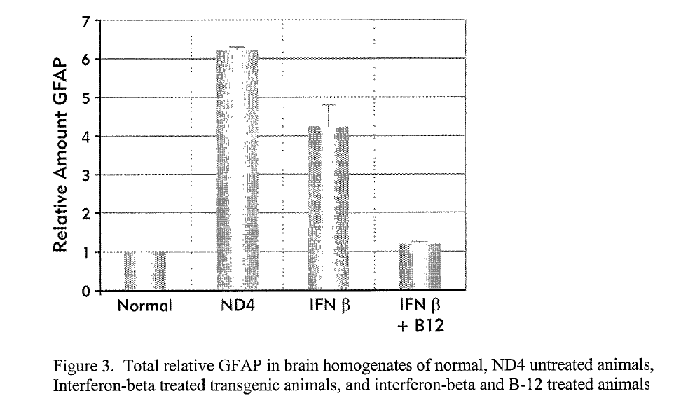

There was a significant increase in GFAP staining of untreated ND4 animals compared to normal animals (FIG. 3), indicating extensive astrogliosis. This high level of GFAP staining was decreased in Interferon-beta treated animals. In addition the image shows high level of inflamation in the white matter tracts. GFAP staining of interferon-beta and B12 treated animals showed significant decrease of inflammation and almost normal GFAP staining compared to the Interferon-beta only treated animals, confirming better efficacy of the Interferon-beta and B12 combination therapy as determined by clinical scores.

### Example 3

GFAP Quantification in Brain Homogenates

Quantification of GFAP within the brains of normal, ND4 untreated and ND4 treated mice was necessary in order to give a more precise level of protein expression (FIG. 4). Whole brain homogenates were assayed for GFAP by slot blot. Brains were homogenized in a buffer containing 50 mM Tris-HCl pH 7.6, 0.5 mM DTT, 1 mM EDTA, and 0.43 mM PMSF. The homogenate was centrifuged at 11000 rpm for 30 min at 4C. the pellet was isolated and resuspended in a buffer containing 10 mM sodium phosphate pH 7.5, 2 mM DTT, 6 M urea and 1 mM EDTA. Samples were loaded onto the slot blot apparatus (BioDot, Biorad) under vacuum. The blots were reacted with anti-GFAP antibodies and then a secondary antibody. The blots were developed and the relative amounts of each band quantified.

Pharmaceutical Compositions

Pharmaceutical compositions of the above compounds are used to treat patients with multiple sclerosis. Vehicles for delivering the compounds of the present invention to target tissues throughout the human body include saline and D5W (5% dextrose and water). Excipients used for the preparation or oral dosage forms of the compounds of the present invention include additives such as a buffer, solubilizer, suspending agent, emulsifying agent viscosity controlling agent, flavour, lactose filler, antioxidant, preservative or dye. There are preferred excipients for parenteral and other administration. These excipients include serum albumin, glutamic or aspartic acid, phospholipids and fatty acids.

The preferred formulation is in liquid form stored in a vial or an intravenous bag. The compounds of the present invention may also be formulated in solid or semisolid form, for example pills, tablets, cream, ointments, powders, emulsions, gelatin capsules, capsules, suppositories, gels or membranes.

The most preferred route of administration is by intravenous injection. Other acceptable routes of administration include conventional oral, topical, rectal, local, inhalant and epidural administration.

In addition to conventional oral drug delivery systems, pharmaceutically acceptable oral controlled release systems that provide a uniform amount of drug at the absorption site include: MacroCap™ (controlled-release pellet system), Micropump™ (oral controlled delivery system), Modas™ (multiporous oral drug absorption system), SCOT™ (single composition osmotic tablet system), Cefom™ microsphere technology, Consurf (constant surface area drug delivery shuttle, Dimatrix™ (diffusion controlled matrix system), DPHS™ (delayed pulsatile hydrogel system), Duredas™ (dual release drug absorption system), GMHS™ (granulating modulating hydrogel system), IPDAS™ (intestitial protective drug absorption system), Pharmazome™ (microparticulate drug delivering technology), PPDS™ (palletized pulsatile delivery system), PRODAS™ (programmable oral drug absorption system), SODAS™ (spheroidal oral drug absorption system), SMHS™ (solubility modulating hydrogel system)

Large-molecule delivery systems utilize technologies for delivering therapeutic biomolecules. Such technologies include: DepoFoam™ system which can be administered by a number of route including intrathecal, subcutaneous, intramuscular, and intraarticular; DUROS™ system which is based on implant technology and Beodas™ (bioerodable enhanced oral drug absorption system), LOCDAS™ (localized drug absoption system) and Micromol System™ that are designed for oral delivery of macromolecules.

Some of the recent transdermal and topical delivery technologies including: Dermaflex™, Dermasite™, D-trans™, Microsponge systems™, TheraDerm-LRS™, Polytrap systems™, Therapatch™.

The compositions of the invention may also be conjugated to transport molecules or included in transport modalities such as vesicles and micelles to facilitate transport of the molecules. Some of the liposomal delivery vesicles include: Novasome lipid vesicles™, Micellar nanoparticles™, Poliposomes™, Stealth liposomes™; INDA™ (insoluble drug absoption systems).

Methods for the preparation of pharmaceutically acceptable compositions that can be administered to patients are known in the art.

The compositions of the invention may also be conjugated to transport molecules, monoclonal antibodies or transport modalities such as vesicles and micelles that preferentially target recipient cells.

Pharmaceutical compositions including the compounds of the present invention can be administered to humans and animals. Dosages to be administered depend on individual patient condition, indication of the drug, physical and chemical stability of the drug, toxicity, the desired effect and on the chosen route of administration (Rakel, R. Ed. 1995). These pharmaceutical compositions are used to treat multiple sclerosis.

Although the invention has been described with preferred embodiments, it is to be understood that modifications may be resorted to as will be apparent to those skilled in the art. Such modifications and variations are to be considered within the purview and scope of the present invention

### REFERENCES

Amit et al.,. Molecular pathogenesis of multiple sclerosis. J. Immunol 100: 252-259, 1999.

Cao et al., Inhibition of experimental allergic encephalomyelitis in the Lewis rat by paclitaxel. J. Neuroummunol. 108:103-111, 2000.

Chofflon et al., Recombinant human interferon beta in relapsing-remitting multiple sclerosis :a review of the major clinical trials. Eur. J. Neur. 7: 369, 2000.

Hajime et al., Clinical usufulness of intrathecal injection of methylcobalamin in patients with diabetic neuropathy. Clin. Ther. 9: 183-187, 1987.

Johnson et al., Copolymer 1 reduces relapse rate and improves disability in relapsing-remitting multiple sclerosis : Results of a phase III mulicenter, double-blind, placebocontrolled trial., Neuro. 45: 1268-1276, 1995;

Johnson et al., Extended use of glatiramer acetate (Copaxone) is well tolerated and maintains its clinical effect on multiple sclerosis relapse rate and degree of disability., Neuro. 50: 701-708, 1998.

Mastronardi, et al. Demyelination in a Transgenic Mouse: A Model for Multiple Sclerosis. 36: 315-324, 1993.

Mattner et al., Inhibition of Th1 development and treatment of chronic-relapsing experimental allergic encephalomyelitis by a non-dihydroxyvitamin D3. Eur. J. Immunol. 30: 498-508, 2000).

Miller et al., Magnetic resonance in monitoring the treatment of multiple sclerosis., Ann Neurol 36: Suppl:S91-4, 1994

Nataf et al., Dihydroxyvitamin D3 Exerts Regional Effects in the Central Nervous System during Experimental Allergic Encephalomyelitis. J. Neuro. Exp. Neuro., 55:904-914, 1996;

O'Connor et al., Ann Neurol 46:470, 1999

Polman et al., Drug treatment of multiple sclerosis., BMJ. 321: 490-494, 2000;

Pouly et al., J. Autoimmun. Multiple sclerosis and central nervous system demyelination. 13: 297-306, 1999;

Rakel, R. Ed. 1995. Conn's Current Therapy. W. B. Saunders Company, U.S.A.

Rudick et al., Disease-Modifying Drugs for Relapsing-Remitting Multiple Sclerosis and Future Directions for Multiple Sclerosis Therapeutics., Arch. Neuro. 56: 1079-1084, 1999;

Rudick et al., Drug Therapy : Management of Multiple Sclerosis., NEJM. 337: 1604-1611, 1997).

Steinman et al., From treatment of experimental allergic encephalomyelitis to clinical trials in multiple sclerosis., Immunol Ser 59: 253-60, 1993;

Tselis et al., Multiple Sclerosis : Therapeutic Update., Arch. Neuro. 56: 277-280, 1999;

Watanabe et al., Ultra-high dose of methylcobalamin promotes nerve regeneration in experimental acrylamide neuropathy. J Neurol. Sci. 122: 140-143, 1994;

Weinstock-Guttman et al., What is new in the treatment of multiple sclerosis?., Drugs 59: 401-410, 2000

Xu et al., Dendritic cell-derived nitric oxide is invovled in IL-4-induced suppression of experimental allergic encephalomyelitis (EAE) in Lewis rats., Clin. Exp. Immunol., 118: 115-121, 1999

Yong et al., Interferon beta in the treatment of multiple sclerosis—Mechanisms of action., Neuro. 51: 682-689, 1998;

We claim:

1. A pharmaceutical composition for the treatment of multiple sclerosis comprising;
(i) a first compound selected from the group consisting of: vitamin B12, analogues of vitamin B12, derivatives of vitamin B12, conjugates of vitamin B12 and mixtures thereof; and
(ii) a second compound selected from the group consisting of interferon-beta, interferon-beta analogues, interferon-beta derivatives, interferon-beta conjugates, and mixtures thereof.

2. A pharmaceutical composition according to claim 1, wherein the first compound is selected from the group consisting of cyanocobalamin (CN-Cb1), aquacobalamin, adenosylcobalamin, methylcobalamin, hydroxycobalamin, cyanocobalamin carbanalide, 5-o-methylbenzylcobalmin [(5-OmeB-za)CN-Cb1], desdimethyl, monoethylamide and methylamide analogues of all of the above, analogues and homologues of cobamamide such as coenzyme B12 and 5-deoxydenosylcobalamin, chlorocobalamin, sulfitocobalamin, nitrocobalamin, thiocyanatocobalamin, benzimidazole derivatives, 5,6-dichlorobenzimidazole, 5-hydroxybenzimidazole, trimethylbenzimidazole, adenosylcyanocobalamin [(Ade) CN-Cb1], cob alamin lactone, cobalamin lactam, anilide, ethylamide, monocarboxylic and dicarboxylic acid derivatives of vitamin B12 or its analogues, mono-, di- and tricarboxylic acid derivatives of vitamin B12, proprionamide derivatives of vitamin B12, polymers of these analogues, and mixtures thereof.

3. A pharmaceutical composition according to claim 2 wherein the first compound is conjugated to the second compound.

4. A pharmaceutical composition according to claim 1 wherein the composition is in injectable form.

5. A pharmaceutical composition according to claim 1 wherein the second compound is selected from the group consisting of interferon beta, AvoneX™, Betaferon™, Rebif™, interferon-beta analogues, pegylated interferon-beta, polymerized interferon-beta, dimerized interferon-beta, interferon-beta as oral inhalant, interferon-beta as an injectable composition, Avonex™ analogues, Betaferon™ analogues, and Rebif™ analogues.

6. A pharmaceutical composition according to claim 1 further comprising a pharmaceutically acceptable carrier.

7. A pharmaceutical composition according to claim 1 further comprising an adjuvant.

8. A method of treating multiple sclerosis comprising the step of administering to a patient the pharmaceutical composition of claim 1.

9. A method of treating multiple sclerosis comprising the following steps:
(i) administering to a patient a pharmaceutical composition comprising a first compound selected from the group consisting of: vitamin B12, analogues of vitamin B12, derivatives of vitamin B12, conjugates of vitamin B12 and mixtures thereof; and
(ii) administering to a patient a pharmaceutical composition comprising a second compound selected from the group consisting of interferon-beta, interferon-beta analogues, interferon-beta derivatives, interferon-beta conjugates, and mixtures thereof.

10. A method according to claim 9 wherein the first compound and the second compound are administered in rapid succession.

11. A method according to claim 9 wherein the first compound is administered in a dose of 2 g to 5 g daily.

12. A method according to claim 9 wherein the amount of the second compound is administered in a dose of 1 μg to 250 μg daily.

13. A method according to claim 9 wherein the first compound is administered one of daily, weekly and monthly and the second compounds is administered one of daily, weekly and monthly.

14. A method according to claim 9 wherein the second compound is selected from the group consisting of: interferon beta, Avonex™, Betaferon™, Rebif™, interferon-beta analogues, pegylated interferon-beta, polymerized interferon-beta, dimerized interferon-beta, interferon-beta as oral inhalant, interferon-beta as injectable compositions, Avonex™ analogues, Betaferon™ analogues, and Rebif™ analogues.
